(19) United States
(12) Patent Application Publication
(10) Pub. No.: US 2006/0230485 A1
(43) Pub. Date: Oct. 19, 2006

(54) See-through mask

(76) Inventor: Sea-Won Lee, Seoul (KR)

Correspondence Address: PARK LAW FIRM, 3255 WILSHIRE BLVD, SUITE 1110, LOS ANGELES, CA, 90010, US

(21) Appl. No.: 10/556,897

(22) PCT Filed: Nov. 4, 2003
(86) PCT No.: PCT/KR03/02345

§ 371 Date: Nov. 14, 2005

(30) Foreign Application Priority Data
May 19, 2003 (KR) 10-2003-0031557

(51) Int. Cl. A61F 9/00

(52) U.S. Cl. 2/15

(57) ABSTRACT

A see-through mask for preventing infection of disease-causing bacteria and inhalation of dusts while providing view of the mask-covered facial area comprises a transparent mask body adapted to cover the nose and the mouth of the user, a wearing instrument for keeping the mask worn on the face of the user, and a respiratory filter provided at a peripheral edge of the mask body for purifying the air inhaled and discharged. The mask enables an observer or dialog partner to see the facial expression of the user, thus assuring comfortable and accurate conversation with the observer.

### TECHNICAL FIELD

The present invention relates to a see-through mask and, more particularly, to a see-through mask for preventing infection of disease-causing bacteria or inhalation of dusts through a respiratory organ.

### BACKGROUND ART

In general, a see-through mask is used to prevent infection of disease-causing bacteria such as viruses through a respiratory organ of a human body or to block introduction of harmful dusts into the human body via the respiratory organ in the worksites where much harmful dusts exist.

There are two types of see-through masks known in the art, i.e., a general type and a high-end type. The general type see-through mask includes a mask body and a filter body made of textile or non-woven fabric in the form of a dual structure. The high-end see-through mask includes a mask body made of synthetic resin such as polyethylene, an air purifying means and an exhaust means mounted through the mask body.

Although the general type see-through mask is cheap, it has shortcomings in that the user has difficulty in breathing and further that humidity is easily generated on an inner surface of the mask. This is because the air has to pass through both the mask body and the filter body during the user's breathing, resulting in poor air ventilation performance.

In the high-end see-through mask, the inhalation and the exhalation of the air are achieved through the air-purifying means and the exhaust means, respectively, so that it has an enhanced air ventilation performance. Therefore, the user has reduced difficulty in breathing and the humidity is hardly generated.

However, the see-through mask described above has drawbacks in terms of the following aspects. When the user wears the mask, the mask body conceals parts of his face (nose, mouth) to make his or her countenance unrecognizable. For this reason, when the user talks with someone, the counterpart tends to have an unpleasant feeling about the user rather than familiarity. Especially, due to the fact that public personnel in, e.g., government offices or a person working in a service field such as hospitals, who have to show an image of kindness, never can show their smiles to citizens or customers, they may give an impression of unkindness to other persons.

Further, in the prior art mask, since the air exhaled from the user is directly emitted to the dialog counterpart, the counterpart may have an unpleasant feeling. Especially, if this situation were in quarantine against contagious disease in an airport or a harbor, the counterpart may try to avoid continuing the dialog.

### DISCLOSURE OF THE INVENTION

It is an object of the present invention to provide a see-through mask allowing an observer or dialog partner to see the facial expression of the wearer.

Another object of the present invention is to provide a see-through mask capable of preventing the air exhaled during the user's breathing from directly reaching the dialog counterpart.

In order to achieve the above objects and other objects, the present invention provides a see-through mask comprising: a mask body adapted to cover a nose and a mouth of a user; a means for keeping the mask body on a face of the user; a filter provided at the mask body for purifying an air inhaled by the user, wherein the mask body is made of a transparent material to allow the face to be seen and the filter is provided at a peripheral edge of the mask body.

### BRIEF DESCRIPTION OF THE DRAWINGS

FIG. 1 shows a frontal view showing an exemplary use of a see-through mask in accordance with one embodiment of the present invention.

FIG. 2 is a side sectional view illustrating another exemplary use of the see-through mask shown in FIG. 1.

FIG. 3 is an exploded perspective view of the see-through mask shown in FIG. 1.

FIG. 4 is a partially enlarged sectional view showing an operation of a discharge valve of the see-through mask shown in FIG. 1.

FIGS. 5, 6 and 7 are frontal views of embodiments of the inventive see-through mask, respectively.

FIG. 8 is a perspective view of another embodiment of the see-through mask of the present invention.

FIG. 9 is an exploded perspective view of the see-through mask shown in FIG. 8.

FIG. 10 is a sectional view of the see-through mask shown in FIG. 8.

FIG. 11 is a sectional view showing an exemplary use of the see-through mask shown in FIG. 8.

### MODES FOR CARRYING OUT THE INVENTION

Preferred embodiments of the present invention will now be described in detail with reference to the accompanying drawings.

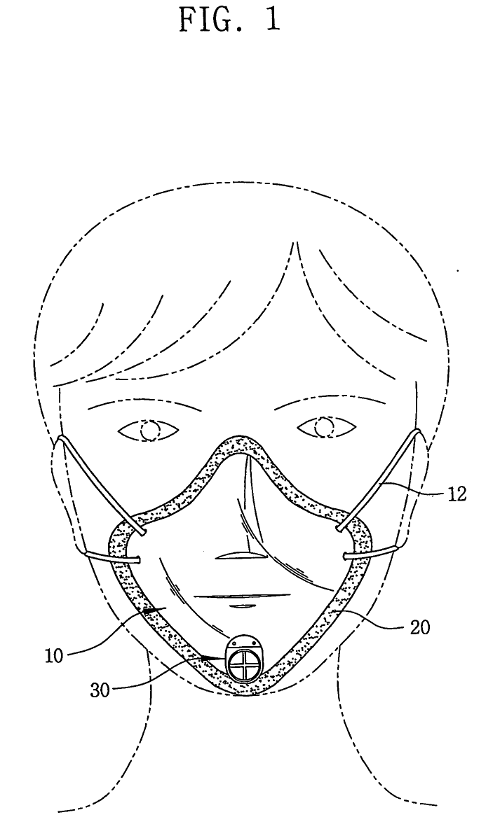

Referring first to FIGS. 1 through 3, the inventive see-through mask includes a mask body 10. The mask body 10 has a shape proper to cover the nose and the mouth of the user and is made of a transparent synthetic resin such as polyethylene, so that the dialog counterpart can see the facial parts (including the nose and the mouth) of the user when the user wears the mask. Further, the mask body 10 is adapted to provide a space between the mask body 10 and the nose and the mouth of the user when he or her wears the mask.

Further, a pair of bands 12 that is expandable and contractible is provided at both ends of the mask body 10 as a wearing instrument for keeping the mask on the user's face. The band 12 is hooked around the ear or the back of the head of the wearer to prevent the mask body 10 from being separated from the face of the wearer. An anti-frost layer 14 is applied on an internal surface of the mask body 10 to prevent forming of frost.

Meanwhile, a means for purifying the air is mounted to the mask body 10. The air-purifying means provides a passageway through which the air is inhaled and functions to remove disease-causing bacteria or fine dusts from the air inhaled, during the breathing of the user wearing the mask body 10. The air-purifying means includes a filter 20 positioned along a peripheral edge of the mask body 10.

The filter 20 is attached to the mask body 10 by an adhesion means like an adhesive along the peripheral edge of the mask body 10. An airtight member 22 made of, e.g., resilient rubber is attached on an external surface of the filter 20 by virtue of an adhesive, etc. The airtight member 22 assures close contact between the face of the wearer and the mask body 10 and allows the wearer to feel good when a contact is made between the wearer and the mask body 10. Especially, the airtight member 22 separates the inside of the mask body 10 from the outside thereof when it is closely contacted with the face of the wearer.

The filter 20 has a folded section 20a, which is folded several times. The folded section 20a maximizes the area of the filter 20 to increase the amount of the inhaled air, magnifying an efficiency of the air purification. Further, since the folded section 20a allows the filter 20 to have a resilient force, it helps assure the close contact between the airtight member 22 and the face of the wearer.

It is preferable to use a HEPA filter as the inventive filter 20. As well known in the art, the HEPA filter is in a paper form obtained from highly fine fiber yarn and is a high-end filter capable of removing particles having a size of 0.3 μm or smaller.

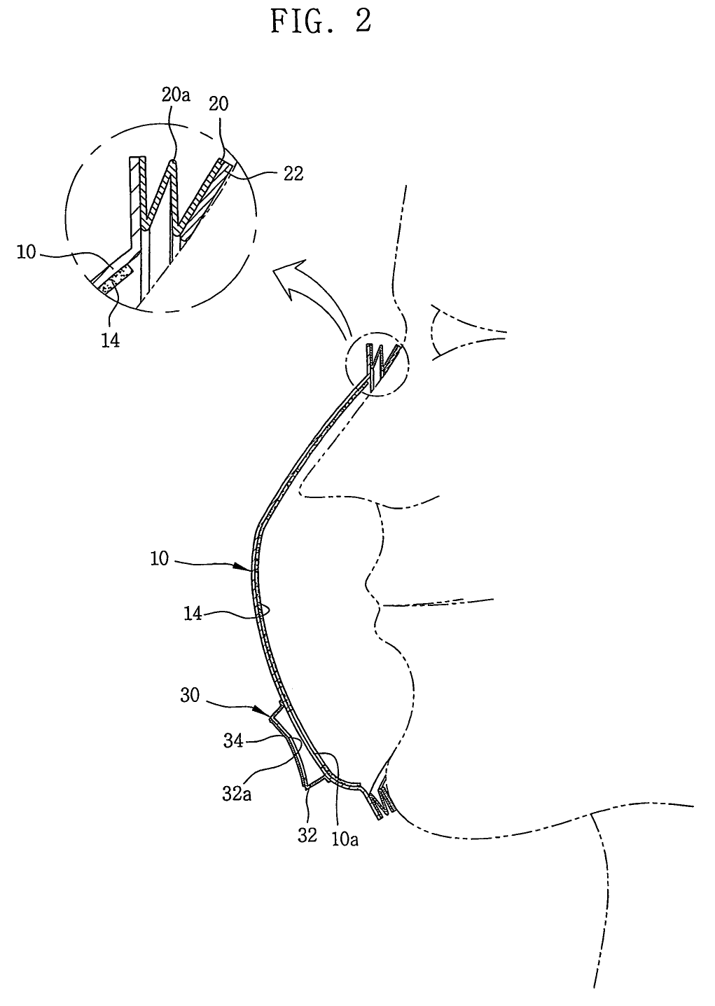

Referring to FIGS. 2 through 4, the mask body 10 includes an exhaust means for discharging the air exhaled from the user. The exhaust means is provided with a discharge hole 10a formed through the mask body 10 and a discharge valve 30 that discharges the air exhaled from the user through the discharge hole 10a and shuts off the discharge hole 10a when the user inhales the air. The discharge hole 10a is formed in a position facing a portion lower than the mouth of the user to properly discharge the air exhaled from the user.

The discharge valve 30 is provided with a valve body 32 that is kept on an external surface of the mask body 10 by an attachment means like a heat melting or an adhesive. The valve body 32 has a valve hole 32a communicating with the discharge hole 10a. The discharge valve 30 is also provided with a flexible valve flap 34 mounted to the valve body 32 to open the valve hole 32a when the user exhales the air and to shut off the valve hole 32a when the user inhales the air. Especially, the flexible valve flap 34 is a thin, flexible plate made of resilient material. The flap 34 is affixed to an external surface of the valve body 32 and initially blocks the valve hole 32a due to its resiliency.

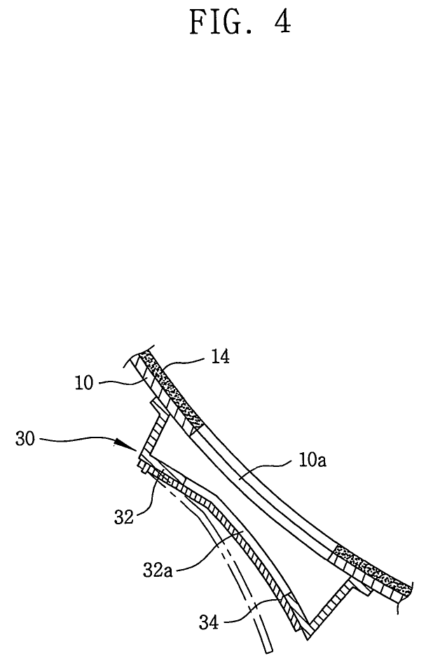

With the exhaust means constructed in this manner, when the wearer exhales the air, the flexible valve flap 34 is deformed as indicated in a phantom line in FIG. 4 to open the valve hole 32a. As a result, the air exhaled from the wearer can be discharged via the valve hole 32a. When the wearer inhales the air, the flexible valve flap 34 is deformed as indicated in a solid line in FIG. 4 to block the valve hole 32a. As a result, the introduction of the outside air into the inside of the mask body 10 is blocked.

The discharge hole 10a and the discharge valve 30 of the valve means are preferably made of transparent materials, so that the face of the wearer can be revealed to the outside.

Next, an exemplary use of the present invention described above will be described with reference to FIGS. 1 and 2. First, when the user wearing the mask with his nose and mouth covered with the mask body 10 inhales the air, a cleaned air is introduced into the inner space of the mask body 10 by the high-end filter 20 that removes the disease-causing bacteria or fine dusts from the air.

When the user exhales the air, the flexible valve flap 34 is deformed as indicated in the phantom line to open the valve hole 32a, thereby allowing the air to be discharged to the outside.

Meanwhile, during the inhaling process of the air, since the discharge valve 30 blocks the valve hole 32a, an introduction of the air via the discharge valve 30 is not permitted. As described above, in the breathing of the wearer, since the introduction of the air is achieved only through the filter 20, while the discharge thereof is achieved only via the discharge valve 30, a ventilation of the air is maintained well without making the wearer feel discomfort in his breathing.

The inventive see-through mask, in which the filter 20 is formed along the peripheral edge of the mask body 10 and the discharge valve 30 is mounted on a lower portion of the mask body 10, allows the nose or the mouth of the wearer to be seen, as shown in FIG. 1, to disclose his real countenance as if he didn't wear the mask.

The anti-frost layer 14 applied on the internal surface of the mask body 10 prevents forming of the frost which may otherwise be formed on the internal surface of the mask body 10 when the wearer breathes or makes a conversation, maintaining a steady transparent condition of the mask body 10.

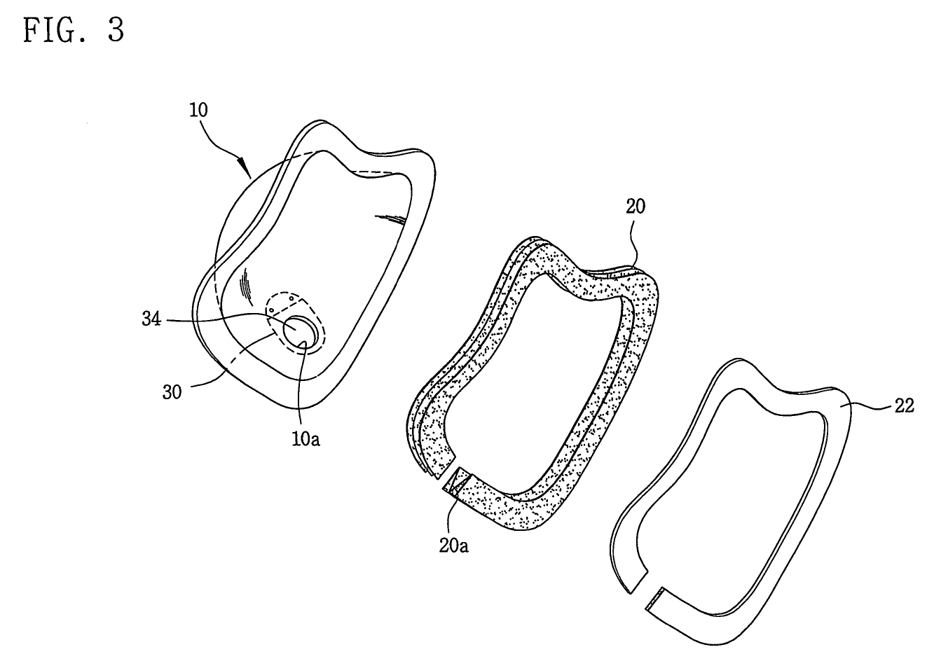

As shown in FIG. 3, since the filter 20 has an increased area due to the folded section 20a, the amount of the air being inhaled and the efficiency of the air-purification can be maximized. Further, since the folded section 20a allows the mask body 10 to move relative to the airtight member 22, the airtight member 22 can be continuously brought into close contact with the face of the wearer in compliance with the face's profile. Further, since the folded section 20a can be deformed in correspondence to the movement of the mouth when the wearer is on a conversation or is laughing, no gap is generated between the airtight member 22 and the face.

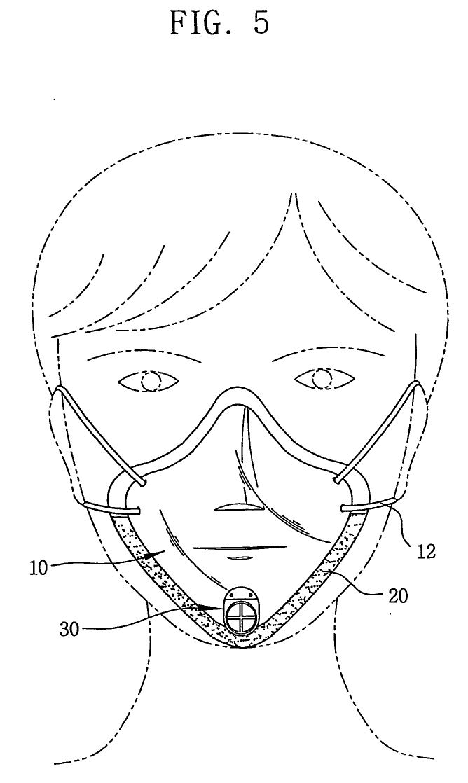

Next, FIG. 5 shows another embodiment of the inventive see-through mask. The see-through mask of this embodiment has the same configuration as the embodiment described above except that the filter 20 for the air-purification is positioned only on a lower area of the mask body 10.

According to this embodiment, since the filter 20 is provided only on the lower area of the mask body 10 along the peripheral edge, those parts of the face hidden by the filter 20 becomes smaller.

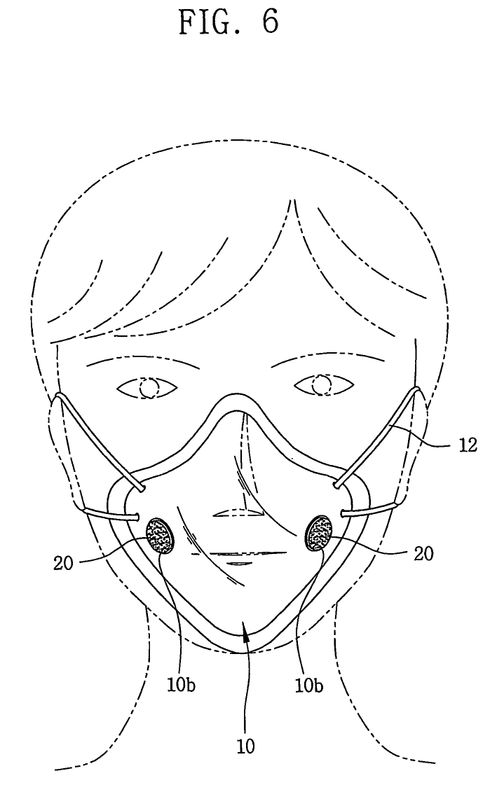

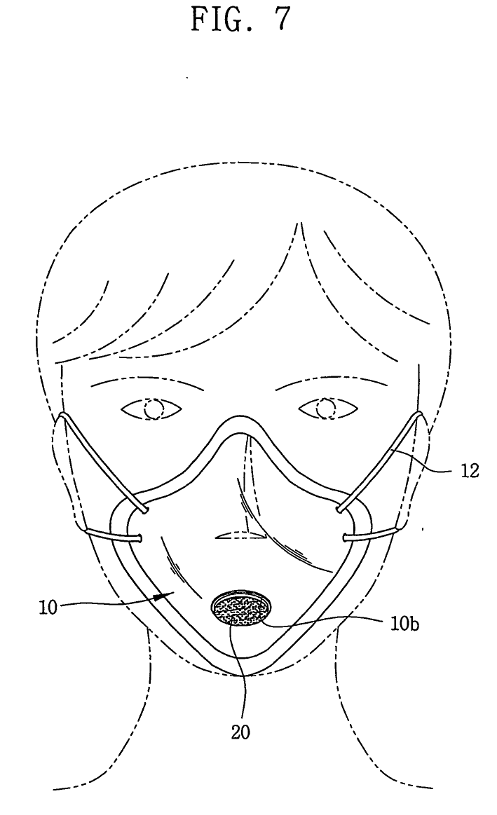

Another embodiment of the inventive see-through mask is shown in FIGS. 6 and 7. In this embodiment, the see-through mask has a filter 20 for the air-purification positioned on particular points of the mask body 10.

To be more specific, through-holes 10b are formed through particular points of the mask body 10 and then the filters 20 are provided at the through-holes 10b, respectively. The see-through holes 10b may be formed through both lateral end portions near the edge of the mask body 10 as shown in FIG. 6 or a lower end near the edge of the mask body 10 as shown in FIG. 7.

With the see-through mask according to this embodiment, it is significantly simple to form the filter 20 by arranging the filter 20 only on the particular points of the mask body 10. Further, unlike the previous embodiment where the filter 20 extends along the edge of the mask body 10, the arrangement of the filter 20 on a position near the nose or the mouth of the wearer ensures that the ventilation is properly performed in response to the wearer's breathing, without having to use a separate air-exhaust means.

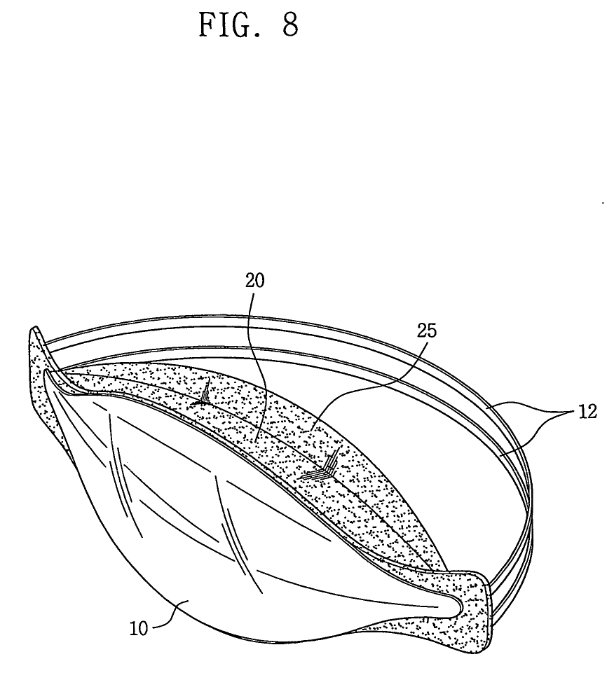

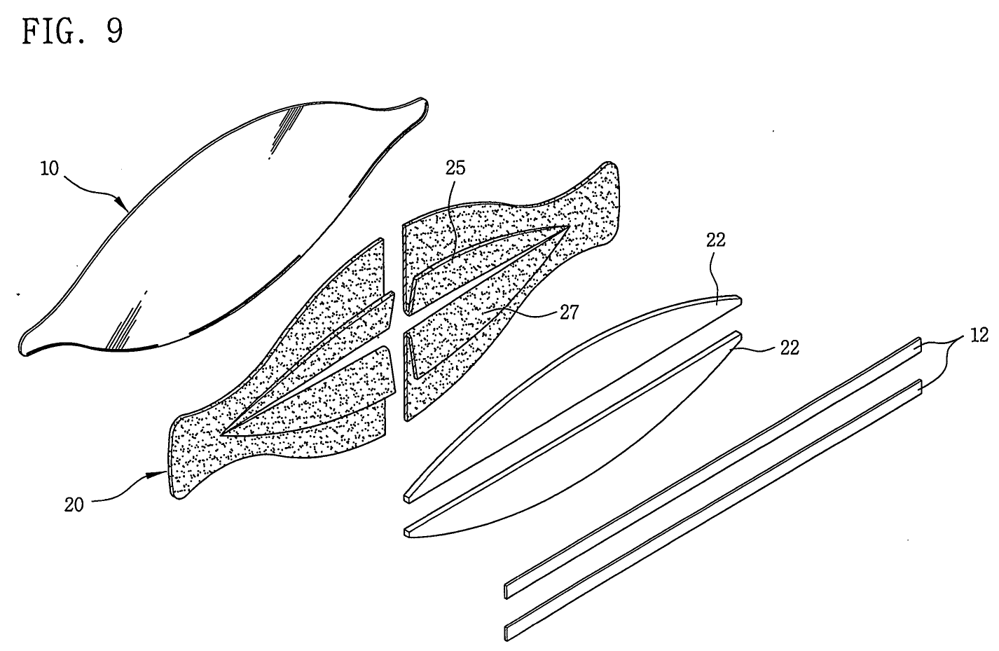

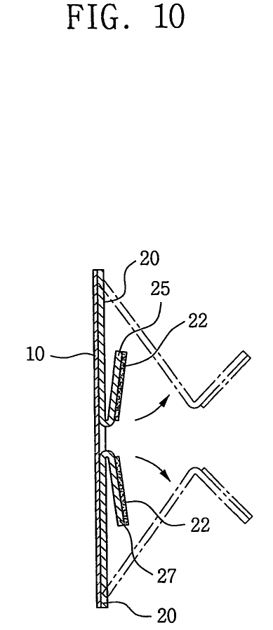

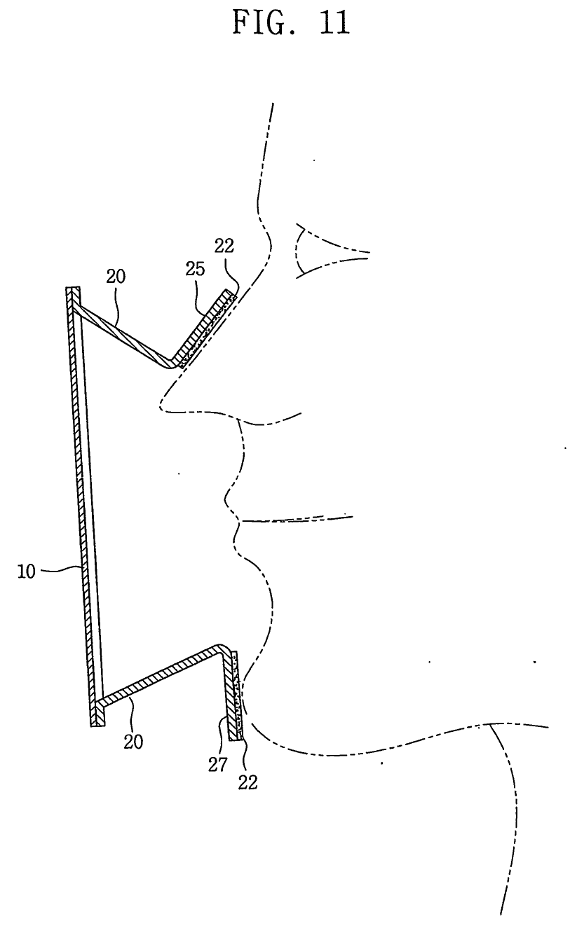

A further embodiment of the inventive see-through mask is shown in FIGS. 8 through 11. The see-through mask according to this embodiment is so configured as to be folded into a flat shape for portability.

More specifically, the see-through mask of this embodiment includes a transparent mask body 10. The mask body 10 is a thin plate like member elongated in a lateral direction and has a size enough to cover the nose and the mouth of the user. The mask body 10 also has an anti-frost layer for preventing the frost from being generated on its inner surface. The filter 20 is, e.g., a HEPA filter made of high fine fiber yarns and affixed to the mask body 10 along its circumference of an edge. The filter 20 can be folded or unfolded with respect to the mask body 10.

An upper portion and a lower portion of the filter 20 have an upper wing 25 foldable upward and a lower wing 27 foldable downward, respectively. The upper wing 25 and the lower wing 27 are symmetrically arranged at the upper portion and the lower portion and come into close contact with the face of the user, especially, those facial portions around the nose and the mouth. The upper wing 25 and the lower wing 27 are equipped with an airtight member 22. The airtight member 22 assures the close contact between the wearer's face and the upper wing and the lower wing 25, 27 and, therefore, the air-tightness between the wearer's face and the filter can be maintained.

Turning to FIG. 8, the see-through mask of this embodiment has a pair of bands 12 that is expandable and contractible. The bands 12 are affixed to both lateral ends of the mask body 10 and are hooked around the back of the head of the wearer.

The see-through mask configured like this is kept on the face of the user by using the pair of bands 12. The mask body 10 is curbed toward lateral ends of the wearer, s face in covering the face. Further, the filter 20 affixed to the mask body 10 is unfolded from the mask body 10 and is closely contacted with the face of the user. Particularly, the upper wing 25 and the lower wing 27 are also unfolded and are closely contacted with the face of the user at the same time.

In this situation, when the wearer breathes, the outside air is introduced into or discharged from an internal space of the mask body 10 via the filter 20.

When the wearer takes off the see-through mask, the mask body 10 is returned to its initial flat form from the curbed form, while the filter 20 is naturally folded toward the mask body 10, making the whole see-through mask flat.

### INDUSTRIAL APPLICABILITY

As described above, in accordance with the inventive see-through mask, thank to the fact that the mask body of the mask is made of a transparent material and the filter for the air-purification is provided in a position in which it does not prevent the nose and the mouth from being seen, the countenance of the wearer can be well expressed. As a result, the wearer can cast familiarity to other people rather than an unpleasant feeling due to the wearing of the mask. Especially, the inventive mask can be advantageously used by the person working in a service field, since he or she can show an image of kindness even though wearing the mask. Further, in a situation of the quarantine against contagious disease, since the countenance of the wearer can be seen, scare or misgivings due to the wearing of the mask may be avoided.

While this invention has been described in connection with what is presently considered to be the most practical and preferred embodiment, it is to be understood that the invention is not limited to the disclosed embodiments, but, on the contrary, is intended to cover various modifications and equivalent arrangements included within the spirit and scope of the appended claims.

What is claimed is:

1. A see-through mask comprising:
a mask body adapted to cover a nose and a mouth of a user;
a means for keeping the mask body on a face of the user; and
a filter provided at the mask body for purifying an air inhaled by the user,
wherein the mask body is made of a transparent material to allow the face to be seen and the filter is provided at a peripheral edge of the mask body.

2. The see-through mask of claim 1, wherein said filter extends along the peripheral edge of the mask body.

3. The see-through mask of claim 2, wherein said filter includes an airtight member made of rubber and capable of making close contact with the face of the user.

4. The see-through mask of claim 2, wherein said filter includes a folded section that is resiliently deformable.

5. The see-through mask of claim 1, wherein said filter is provided on a particular point of the peripheral edge of the mask body.

6. The see-through mask of claim 1, wherein an anti-frost layer is formed on an inner surface of the mask body.

7. The see-through mask of claim 1, further comprising an exhaust means, wherein the exhaust means comprises:
a discharge hole formed through the mask body;
a valve body having a valve hole communicating with the discharge hole and mounted to-the mask body; and
a flexible valve flap affixed to the valve body to open the valve hole when the user exhales the air and to shut off the valve hole when the user inhales the air.

8. The see-through mask of claim 1, wherein said mask body comprises a transparent plate member having an initially flat form and being curved in compliance with a profile of the face when the user wears the mask and said filter is foldably attached to the peripheral edge of the mask body.

9. The see-through mask of claim 8, wherein an anti-frost layer is formed on an inner surface of the mask body.
